(19) United States
(12) Patent Application Publication
(10) Pub. No.: US 2017/0203286 A1
(43) Pub. Date: Jul. 20, 2017

(54) IONIC LIQUID, ADDUCT AND METHODS THEREOF

(71) Applicant: RELIANCE INDUSTRIES LIMITED, Mumbai, Maharashtra (IN)

(72) Inventors: Parasuveera UPPARA, Navi Mumbai (IN); Vivek RAJE, Washim (IN); Pavankumar ADURI, Dombivali (IN); Vibhuti DUKHANDE, Mumbai (IN); Prathmesh Pradeep SALVI, Dist. Ratnagiri (IN)

(73) Assignee: RELIANCE INDUSTRIES LIMITED, Mumbai, Maharashtra (IN)

(21) Appl. No.: 15/324,785

(22) PCT Filed: Jul. 9, 2015
(86) PCT No.: PCT/IB2015/055196

§ 371 Date: Jan. 9, 2017

(30) Foreign Application Priority Data
Jul. 10, 2014 (IN) 2257/MUM/2014

(51) Int. Cl. B01J 31/02; B01J 37/00; C07C 37/11; C07C 2/68; C07C 45/46; C07F 5/06; C07C 2/22; C07C 2/86; B01J 37/04; C07C 67/293

(52) U.S. Cl. CPC B01J 31/0298; B01J 31/0287; B01J 37/04; B01J 37/009; C07C 37/11; C07C 67/293; C07C 45/46; C07F 5/06; C07C 2/22; C07C 2/861; C07C 2/68; B01J 2531/004; B01J 2231/44; B01J 2231/20; B01J 2231/326; C07C 2527/126; C07C 2531/02

(57) ABSTRACT

The present disclosure relates to preparation of liquid salt including but not limiting to ionic liquid and applications thereof. More particularly, the present disclosure provides a process for preparing ionic liquid which comprises reacting at least one electron-pair acceptor and at least one electron-pair donor to form an adduct, and reacting the adduct with at least one electron-pair acceptor to prepare said salt. The present disclosure also provides for applications of the ionic liquid prepared in the present disclosure.

### TECHNICAL FIELD

The present disclosure relates to organic chemistry in general and reactions of organic compounds in particular. The present disclosure provides a process for preparing liquid salt including but not limiting to ionic liquid. More particularly, the present disclosure relates to a process which involves reacting at least one electron-pair acceptor and at least one electron-pair donor to form an adduct. The adduct is in-turn reacted with at least one electron-pair acceptor to prepare said liquid salt of the present disclosure. The process of the present disclosure thus provides ionic liquid without subjecting the reactants to heating. The present disclosure also relates to applications of the ionic liquid of the present disclosure across varied organic reactions.

### BACKGROUND

Salts are ionic compounds that result from the neutralization reaction of an acid and a base. They are composed of related numbers of cations (positively charged ions) and anions (negative ions) so that the product is electrically neutral (without a net charge). These component ions can be inorganic or organic, and salts as a whole can be monatomic, or polyatomic. Salts may be in solid form or liquid form, and salts in liquid state are known as ionic liquids.

Ionic liquids are thus liquids that are composed entirely of ions or a combination of cations and anions. The so-called “low temperature” Ionic liquids are generally organic salts with melting points less than 100 degrees C., often even lower than room temperature. Ionic liquids may be suitable, for example, for use as catalysts and solvents in alkylation and polymerization reactions as well as in dimerization, oligomerization acetylation, metatheses and copolymerization reactions.

One class of ionic liquids is fused salt compositions, which are molten at low temperature and are useful as catalysts, solvents and electrolytes. Such compositions are mixtures of components which are liquids at temperatures below the individual melting points of the components. The most common ionic liquids are those prepared from organic-based cations and inorganic or organic anions. The most common organic cations are ammonium cations. Ionic liquids of pyridinium and imidazolium are perhaps the most commonly used cations. Anions include, but are not limited to BF4-, PF6-, haloaluminates such as Al2Cl7- and Al2Br7-, [(CF3 SO2)2N)]-, alkyl sulphates (RSO3-), carboxylates (RCO2-) and many others. The most catalytically interesting ionic liquids are those derived from ammonium halides and Lewis acids (such as AlCl3, TiCl4, SnCl4, FeCl3 and the like). Chloroaluminate ionic liquids are perhaps the most commonly used ionic liquid catalyst systems.

The Lewis acids, which are electron pair acceptors, and Lewis bases, which are electron pair donors, react to form adducts in which a coordinate covalent bond is formed. This type of bond is usually represented by an arrow. The strength of the interaction between a Lewis acid and a Lewis base is controlled by at least two factors, electronic and steric. Electron donating groups on an atom can increase the Lewis basicity of that atom, while electron-withdrawing groups can increase the Lewis acidity.

Often metal complexes expand their coordination number by interaction with a Lewis base. This may take place by intermolecular association or by adduct formation with solvent or available ligands of comparable ligating ability. The physical properties of the resulting complex often are significantly different from those of the complex not having the expanded coordination number. The ability to interact with bases seems to be related closely to the electronic properties of the ligands as a whole, not just the atoms bonded to the metal.

WO/2011/064556 discloses formation of a mixture having a freezing point up to 100° C. formed by contacting 1 mole of AlX3, where X can be CL, Br, F with 1 or 2 moles of R1-C(O)—N(R2)(R3) where R1 to R3 can be alkyl, Aryl or substituted alkyl and aryl. This mixture can be used for electroreduction of the mixture to produce aluminium metal. It also discloses the solid formation of AlX₃ with 3 moles of Amide. However, this mixture requires heating to form a good mixture, having freezing point up to 100° C.

As per the methods of the prior art, addition of Lewis acid to weak Lewis base requires heat to initiate the reaction to form ionic liquid. Thus, the methods of the prior art are cumbersome and require excessive heating to obtain the ionic liquids. Further, the ionic liquids so obtained by the method of the prior arts are highly viscous, thereby making them difficult to handle, transfer, pump in reaction vessel and employ in industrial processes. The present disclosure aims to overcome the drawbacks observed in the currently available technology and provides for an easy and convenient method to prepare ionic liquids.

### SUMMARY OF THE DISCLOSURE

The present disclosure relates to a process of preparing salt such as liquid salt preferably ionic liquid by reacting at least one electron-pair acceptor and at least one electron-pair donor to form an adduct, and further reacting the adduct with at least one electron-pair acceptor to prepare said salt.

The present disclosure relates to a method of preparing ionic liquid, said method comprising acts of contacting at least one electron-pair acceptor with at least one electron-pair donor to obtain an adduct and contacting the adduct with at least one electron-pair acceptor to obtain the ionic liquid.

In various embodiments, the present disclosure relates to a process of preparing liquid salt preferably ionic liquid by reacting at least one electron-pair acceptor or Lewis acid and at least one electron-pair donor or Lewis base to form an adduct. The adduct is thereafter further reacted with at least one electron-pair acceptor or Lewis acid to prepare said liquid salt.

The present disclosure also relates to ionic liquid prepared by the process of the present disclosure, which comprises contacting at least one electron-pair acceptor with at least one electron-pair donor to obtain an adduct; and contacting the adduct with at least one electron-pair acceptor to obtain the ionic liquid. In a non-limiting embodiment, the process of the present disclosure is carried out without subjecting the reactants to heating. In another non-limiting embodiment of the present disclosure, the electron-pair donor is not an amine.

The present disclosure also relates to applications of the liquid salt including but not limiting to ionic liquid prepared by the process of the present disclosure. In an embodiment, the ionic liquid is suitable for applications involving organic reactions including but not limiting to catalysis, alkylation, trans-alkylation, acylation, polymerization, dimerization, oligomerization, acetylation, metatheses, pericyclic and copolymerization reactions.

The present disclosure, also relates to a method of preparing an adduct of electron-pair acceptor and electron-pair donor, said method comprising act of contacting at least one electron-pair acceptor with at least one electron-pair donor to obtain the adduct.

The present disclosure also relates to adduct prepared according to the process of the present disclosure, which comprises act of contacting at least one electron-pair acceptor with at least one electron-pair donor to obtain the adduct. In an embodiment, the said adduct is capable of forming ionic liquid on further reacting with electron-pair acceptor.

### DETAILED DESCRIPTION

The present disclosure relates to a method of preparing ionic liquid, said method comprising acts of:
a) contacting at least one electron-pair acceptor with at least one electron-pair donor to obtain an adduct; and
b) contacting the adduct with at least one electron-pair acceptor to obtain the ionic liquid.

The present disclosure also relates to an ionic liquid prepared according to the aforesaid method.

The present disclosure also relates to use of the aforesaid ionic liquid for application in chemical reaction.

The present disclosure also relates a method of preparing an adduct of electron-pair acceptor and electron-pair donor, said method comprising act of contacting at least one electron-pair acceptor with at least one electron-pair donor to obtain the adduct.

The present disclosure also relates an adduct prepared accordingly to the above method.

In an embodiment of the present disclosure, the method of preparing the ionic liquid comprises acts of:
a) contacting at least one electron-pair acceptor with at least one electron-pair donor, in presence or absence of first solvent, to obtain a mixture;
b) optionally mixing and filtering the mixture of step (a) to obtain a filtrate and optionally washing the filtrate or the mixture of step (a) with a second solvent, followed by drying to obtain the adduct; and
c) contacting the adduct of step (b) with at least one electron-pair acceptor in presence or absence of third solvent, followed by mixing to obtain the ionic liquid.

In an embodiment of the present disclosure, the method of preparing the adduct comprises acts of:
a) contacting at least one electron-pair acceptor with at least one electron-pair donor, in presence or absence of first solvent, to obtain a mixture; and
b) optionally mixing and filtering the mixture of step (a) to obtain a filtrate and optionally washing the filtrate or the mixture of step (a) with a second solvent, followed by drying to obtain the adduct.

In another embodiment of the present disclosure, the step a) or step b) or a combination thereof is carried out in presence of solvent; the electron acceptor used in step b) is same as or different than that used in step a); addition of the solvent is carried out along with mixing; ratio of the electron-pair acceptor to the electron-pair donor in step a) is ranging from about 1:1 to about 1:5; concentration of the adduct in step b) is ranging from about 0.001 mol to about 0.9 mol; and ratio of the adduct to the electron-pair acceptor in step b) is ranging from about 1:1 to about 1:6.

In yet another embodiment of the present disclosure, the method is carried out in presence of solvent; wherein addition of the solvent is carried out along with mixing; and wherein ratio of the electron-pair acceptor to the electron-pair donor is ranging from about 1:1 to about 1:5.

In still another embodiment of the present disclosure, the method of preparing the ionic liquid is carried out in absence of heating; the method is carried out under inert atmosphere; and wherein the inert atmosphere is Nitrogen atmosphere.

In still another embodiment of the present disclosure, the electron acceptor is a salt of cation selected from group comprising aluminium, magnesium, calcium, chromium, manganese, iron, cobalt, nickel, copper, zinc, gallium, germanium, zirconium, scandium, vanadium, molybdenum, ruthenium, rhodium, indium, tin, titanium, lead, cadmium and mercury or any combination thereof; the electron acceptor is a salt of cation selected from group comprising acetate, carbonate, chloride, citrate, cyanide, fluoride, nitrate, nitrite, phosphate and sulfate or any combination thereof; and the concentration of the electron acceptor is ranging from about 0.001 mol to about 0.9 mol.

The method as claimed in claim 1 or claim 4, wherein the electron donor is not an amine; the electron donor is selected from group comprising phosphine, amide, alkyl sulfoxide, ester and alcohol or any combination thereof; the phosphine is selected from group comprising triphenylphosphine, triphenylphosphine oxide, trimethylphosphine and tributylphosphine or any combination thereof; the amide is selected from group comprising urea, dimethyl formamide, acetamide, N-methyl pyrrolidine, thiourea, phenylthiourea, acetanilide, propanamide, 3-methylbutanamide, dimethylacetamide and butanamide or any combination thereof; the alkyl sulfoxide is dimethyl sulfoxide; the ester is selected from group comprising amyl acetate, ethyl acetate and propyl acetate or any combination thereof; the alcohol is cyclohexanol and isopropyl alcohol or any combination thereof; and the concentration of the electron donor is ranging from about 0.001 mol to about 0.9 mol.

In still another embodiment of the present disclosure, the first solvent, the second solvent or the third solvent are same or different; wherein the solvent is selected from group comprising Ethyl Acetate, Benzene, Toluene, Ethanol, Acetic Acid, Acetonitrile, Butanol, Carbon Tetrachloride, Chlorobenzene, Chloroform, Cyclohexane, 1,2-Dichloroethane, Heptane, Hexane, Methanol, Methylene Chloride, Nitromethane, Pentane, Propanol and Xylene or any combination thereof; and wherein the amount of the solvent is ranging from about 1% to about 80%.

In still another embodiment of the present disclosure, the solvent in step a) is added to either the electron-pair acceptor or the electron-pair donor, prior to the said contacting; wherein the contacting is carried out along with mixing; wherein the mixing is carried out for a time duration ranging from about 1 minute to about 12 hours, at a temperature ranging from about 5° C. to about 50° C.; and wherein the mixing is carried out by technique selected from group comprising stirring, milling, blending, static mixing, and grinding, or any combination thereof.

In still another embodiment of the present disclosure, wherein the chemical reaction is selected from group comprising catalysis, alkylation reaction, trans-alkylation reaction, acylation reaction, polymerization reaction, dimerization reaction, oligomerization reaction, acetylation reaction, metatheses reaction, pericyclic reaction and copolymerization reaction or any combination thereof.

In an embodiment of the present disclosure, the terms ‘catalyst’, ‘ionic liquid’, ‘ionic liquid catalyst’ and ‘ionic catalyst’ are used interchangeably.

The present disclosure relates to a process of preparing salt by reacting at least one electron-pair acceptor and at least one electron-pair donor to form an adduct, and further reacting the adduct with at least one electron-pair acceptor to prepare said salt.

In a non-limiting embodiment, the present disclosure relates to a process of preparing salt preferably liquid salt including but not limiting to ionic liquid by reacting at least one electron-pair acceptor and at least one electron-pair donor to form an adduct, and further reacting the adduct with at least one electron-pair acceptor to prepare said liquid salt.

In a preferred embodiment, the electron-pair acceptor employed in the process of preparing liquid salt including but not limiting to ionic liquid is a Lewis acid and the electron-pair donor employed in said process is a Lewis base. Thus, the present disclosure provides a process of preparing liquid salt including but not limiting to ionic liquid by reacting at least one Lewis acid with at least one Lewis base to form an adduct, which is further reacted with at least one Lewis acid to prepare said liquid salt.

In a non-limiting embodiment, the process of the present disclosure for preparing liquid salt including but not limiting to ionic liquid is carried out without subjecting the reactants to heating.

In an embodiment of the present disclosure, use of even a weak Lewis base, such as but not limiting to urea, for formation of ionic liquid through an intermediary adduct, does not require subjecting the reactants to heating.

In a non-limiting embodiment of the present disclosure, the Lewis acid reacted with the adduct in the process of the present disclosure is the same Lewis Acid which is reacted initially with the Lewis base to form the adduct. In another non-limiting embodiment of the present disclosure, the Lewis acid reacted with the adduct in the process of the present disclosure is different from the Lewis Acid which is reacted initially with the Lewis base to form the adduct.

In an embodiment, addition of electron-pair acceptor (Lewis acid) to electron-pair donor (Lewis base) reacts to form adducts in which a coordinate covalent bond is formed. This type of bond is usually represented by an arrow. The strength of interaction between an electron-pair acceptor and an electron-pair donor is controlled by at least two factors, electronic and steric. Electron donating groups on an atom increases the Lewis basicity of that atom, while electron-withdrawing groups increases the Lewis acidity. Metal complexes expand their coordination number by interacting with a Lewis base. This takes place by intermolecular association with solvent or by adduct formation with solvent or availability of ligands of comparable ligating ability. The physical properties of the resulting complex often are significantly different from those of the complex not having the expanded coordination number. ML4 (M=metal; L=ligand) complexes vary substantially in their ability to interact with Lewis bases. The ability to interact with bases is related closely to the electronic properties of the ligands as a whole, not just the atoms bonded to the metal.

M1Xₘ+nLB→[M1(LB)ₙXₘ]

Wherein M1 is metal, X is halide, LB is Lewis Base, n depends on the coordination capability of M1. M1 can be Cu, Zn, Fe, Al, Ga, In, Zr, Sc, Ti, V, Ca, Mg, Mn, Co, Ru, Rh, Sn, Pb, Mb, Hg, etc

In an embodiment, adduct formation depends on type of metal and the ligand attached to it. For instance, CaCl₂ forms an adduct with 2 electron donors, whereas AlCl₃ forms an adduct with 3 electron donors. The present disclosure is based on the theory of co-ordinate bonds between filled orbitals of electron rich molecule overlapping the empty orbitals of the electron deficient molecules. π electrons involving in such bonding, polarize the molecule inheriting the acidic sites, favourable for chemical reactions such as alkylation.

In an embodiment of the present disclosure, different types of Ionic Liquid are formed by varying the Metal salt as well as Lewis Base employed. In an exemplary embodiment, the Ionic liquid obtained is:
1) Acidic IL: such as AlCl₃-UREA: +AlCl₃
2) Basic IL: such as MgCl₂-UREA+MnCl₂
3) Neural IL: such as ZnCl₂-UREA+ZnCl₂

In an embodiment, the ionic liquid of the present disclosure is formed by reacting an electron-pair acceptor with an electron-pair donor in a particular order.

In an embodiment, the adduct of electron-pair acceptor and electron-pair donor is stabile and leads to the formation of ionic liquid. In an embodiment, the stability is directly proportional to the ratio between the Lewis base and Lewis acid. The adduct stability is also dependent on the type of electron pair donor as well as electron pair acceptor.

In an embodiment of the present disclosure, the ionic liquid is formed by reacting the adduct of Lewis base and Lewis acid with Lewis acid, and not by reacting a salt of Lewis Base and Bronsted acid with Lewis Acid. A Bronsted acid forms a salt and not an adduct with a Lewis base.

For formation of ionic liquid from a salt of Lewis base and Bronsted acid, such as 1-Butyl-3-methylimidazolium Chloride ([BMIM][Cl]), the salt is reacted with a metal halide, such as AlCl₃, in a salt: AlCl₃ ratio of 1:1 and up to 1:3. If said ratio is greater than 1:3, the AlCl₃ starts precipitating out from the ionic liquid formed and hence concentration of the AlCl₃ in the ionic liquid formed should not be beyond three times the concentration of the salt. However, as is the case in the present disclosure, when Ionic Liquid is formed from the adduct, which in-turn is formed from electron pair acceptor (metal halide such as AlCl₃) and electron pair donor, will require about 3 moles of AlCl₃ and once the ionic liquid is formed it can further take about 3 more moles of AlCl₃ making the ratio of adduct:AlCl₃ to about 1:6. Thus, higher concentration of AlCl₃ per mole of adduct is dissolved in the ionic liquid formed through an adduct than when formed through a salt.

In an embodiment, the adduct based ionic liquids of the present disclosure, can dissolve more metal halide. Due to this property the adduct-based ionic liquid are also useful in applications for metal deposition. Further, the adduct based ionic liquid have higher activity as a catalyst due to presence of high active catalyst.

In an embodiment of the present disclosure, formation of the adduct is done by reacting one Lewis acid with one Lewis Base, one Lewis acid with two Lewis Base, one Lewis acid with three Lewis bases, one Lewis acid with four Lewis bases, and so on, depending on the vacant orbitals on the central metal atom of the Lewis Acid. In an embodiment of the present disclosure, an adduct is formed between the electron-pair acceptor (Lewis acid) and electron-pair donor (Lewis base) when ratio of the electron-pair acceptor to the electron-pair donor is ranging from about 1:1 to about 1:5

In an embodiment, addition of more Lewis acid to the adduct formed results in collapse in the structure of the adduct which leads to formation of the Ionic Liquid.

In an embodiment of the present disclosure, an ionic liquid is formed between the aforesaid adduct and the electron-pair acceptor (Lewis acid) when ratio of the adduct to the electron-pair acceptor is ranging from about 1:1 to about 1:6.

In an embodiment, the ionic liquid of the present disclosure is stable, has good thermal conductivity, biocompatibility and increased active surface area. It has several applications industrially and in other areas. In an embodiment, the ionic liquids of the present disclosure can be regenerated and recycled.

In a non-limiting embodiment, the Lewis acid employed in the present disclosure is a salt of cation including but not limiting to Aluminium (Al), Magnesium (Mg), Calcium (Ca), Chromium (Cr), Manganese (Mn), Iron (Fe), Cobalt (Co), Nickel (Ni), Copper (Cu), Zinc (Zn), Gallium (Ga), Germanium (Ge), Indium (In), Zirconium (Zr), Scandium (Sc), Vanadium (V), Molybdenum (Mb), Ruthenium (Ru), Rhodium (Rh), Tin (Sn), Titanium (Ti), Lead (Pb), Cadmium (Cd) and Mercury (Hg).

In a non-limiting embodiment, the Lewis acid employed in the present disclosure is a salt of cation, wherein the anionic moiety of the salt includes but is not limited to inorganic, organic, monatomic and polyatomic moiety. In an exemplary embodiment, the anion moiety in the salt includes but is not limited to Acetate, Carbonate, Chloride, Citrate, Cyanide, Fluoride, Nitrate, Nitrite, Phosphate and Sulfate.

In a non-limiting embodiment, the Lewis base employed in the present disclosure includes but is not limited to, In a non-limiting embodiment, the Lewis base employed in the present disclosure is selected from a group comprising phosphine class of compound, amide class of compound, alkyl sulfoxide class of compound, ester class of compound and alcohol class of compound or any combination thereof. In an exemplary embodiment of the present disclosure, the phosphine class of compound is selected from a group comprising triphenylphosphine, triphenylphosphine oxide and tributylphosphine or trimethylphosphine (PMe3) or any combination thereof; the amide class of compound is selected from a group comprising urea, dimethyl formamide (DMF), acetamide, N-methyl pyrrolidine (NMP), thiourea, phenylthiourea, acetanilide, propanamide, 3-methylbutanamide and butanamide, Dimethylacetamide (DMA) or any combination thereof; alkyl sulfoxide class of compound is dimethyl sulfoxide (DMSO); ester class of compound is selected from a group comprising amyl acetate and propyl acetate and ethyl acetate (EtOAc) or any combination thereof; the alcohol is selected from a group comprising cyclohexanol and isopropyl alcohol (IPA) or combination thereof.

In a preferred embodiment, the process of the present disclosure is carried out without employing amine as the Lewis base.

Amine is toxic and has slow biodegradability when compared to other Lewis bases such as amides, alcohols, esters, etc. Hence, use of amine for formation of ionic liquid is avoided to make the ionic liquids of the present disclosure more user and environment friendly.

In an exemplary embodiment, the process of preparing liquid salt including but not limiting to ionic liquid in the present disclosure comprises acts of:
a) contacting at least one electron-pair acceptor with at least one electron-pair donor in presence or absence of a solvent to obtain an adduct; and
b) Contacting the adduct with at least one electron-pair acceptor in presence or absence of a solvent to prepare the liquid salt of the present disclosure.

In a preferred embodiment, the at least one electron-pair acceptor is contacted with the at least one electron-pair donor in presence of a solvent under nitrogen atmosphere to obtain a slurry. The slurry is stirred and thereafter subjected to filtration, followed by washing with a solvent to obtain an adduct.

The adduct is contacted with the at least one electron-pair acceptor in presence of a solvent under nitrogen atmosphere to obtain a mass. The mass is further stirred to obtain the liquid salt of the present disclosure.

In an exemplary embodiment, the process of preparing liquid salt including but not limiting to ionic liquid in the present disclosure comprises acts of:
a) contacting at least one electron-pair acceptor with a first solvent under nitrogen atmosphere to obtain a mixture;
b) adding at least one electron-pair donor to the mixture of step (a) to obtain a slurry;
c) stirring and filtering the slurry of step (b) and washing with a second solvent followed by drying to obtain an adduct;
d) contacting the adduct of step (c) with a third solvent under nitrogen atmosphere, followed by stirring, to obtain a mixture; and
e) adding at least one electron-pair acceptor to the mixture of step (d), followed by stirring to prepare the liquid salt.

In a preferred embodiment, the at least one electron-pair donor is added to the mixture under stirring to obtain a slurry. In another preferred embodiment, the adduct is contacted with a solvent under stirring to obtain a mixture, and thereafter, the mixture is subjected to water bath before addition of the at least one electron-pair acceptor.

In another preferred embodiment, the electron-pair acceptor employed in the process of preparing liquid salt including but not limiting to ionic liquid is a Lewis acid and the electron-pair donor employed in said process is a Lewis base. In a non-limiting embodiment of the present disclosure, the Lewis acid reacted with the adduct in the process of the present disclosure is the same Lewis Acid which is reacted initially with the Lewis base to form the adduct. In another non-limiting embodiment of the present disclosure, the Lewis acid reacted with the adduct in the process of the present disclosure is different from the Lewis Acid which is reacted initially with the Lewis base to form the adduct.

In a non-limiting embodiment, the adduct of the method of the present disclosure is directly exposed to nitrogen atmosphere without the third solvent.

In a non-limiting embodiment, the electron-pair acceptor or the Lewis acid employed in the process of preparing liquid salt including but not limiting to ionic liquid is present at an amount ranging from about 0.001 mol to about 0.9 mol, preferably about 0.3 mol to about 0.8 mol.

In a non-limiting embodiment, the electron-pair donor or the Lewis base employed in the process of preparing liquid salt including but not limiting to ionic liquid is present at an amount ranging from about 0.001 mol to about 0.9 mol, preferably about 0.3 mol to about 0.8 mol.

In a non-limiting embodiment, the adduct employed for reaction with at least one electron-pair acceptor to prepare liquid salt of the present disclosure is present at an amount ranging from about 0.01 mol to about 0.9 mol, preferably from about 0.1 mol to about 0.7 mol.

In a non-limiting embodiment, the process of preparing liquid salt including but not limiting to ionic liquid in the present disclosure comprises acts of:
a) contacting at least one electron-pair acceptor at an amount ranging from about 0.001 mol to about 0.9 mol with a first solvent under nitrogen atmosphere to obtain a mixture;
b) adding at least one electron-pair donor at an amount ranging from about 0.001 mol to about 0.9 mol to the mixture of step (a) under stirring to obtain a slurry;
c) stirring and filtering the slurry of step (b) and washing with a second solvent followed by drying to obtain an adduct;
d) contacting the adduct of step (c) at an amount ranging from about 0.001 mol to about 0.9 mol with a third solvent under nitrogen atmosphere, followed by stirring, to obtain a mixture; and
e) adding at least one electron-pair acceptor at an amount ranging from about 0.001 mol to about 0.9 mol to the mixture of step (d) under stirring, followed by further stirring to prepare the liquid salt.

In a non-limiting embodiment, the at least one electron-pair donor is added to the mixture of step (a) of the process of the present disclosure for a time period ranging from about 1 minute to 60 minutes at a temperature ranging from about 10° C. to about 50° C.

In a non-limiting embodiment, the stirring of the slurry of the process of the present disclosure is carried out for a time period ranging from about 1 hour to about 10 hours.

In another non-limiting embodiment, the adduct is contacted with a solvent under stirring to obtain a mixture, and thereafter, the mixture is subjected to water bath having temperature ranging from about 30° C. to about 50° C., before addition of the at least one electron-pair acceptor.

In another non-limiting embodiment, the at least one electron-pair acceptor is added to the mixture of step (d) of the process of the present disclosure under stirring for a time period ranging from about 1 minute to 60 minutes. In another non-limiting embodiment, the further stirring of the mixture to prepare the liquid salt is carried out for a time period ranging from about 1 hour to about 10 hours.

In another non-limiting embodiment of the present disclosure, the solvent initially contacted with the at least one electron-pair acceptor (first solvent), the solvent employed for washing the slurry of the present disclosure (second solvent) and the solvent contacted with the at least one electron-pair acceptor to obtain the liquid salt of the present disclosure (third solvent) are either all same, or all different or a combination thereof.

In a non-limiting embodiment, the process of preparing liquid salt of the present disclosure is carried out in presence of a solvent, preferably organic solvent including but not limiting to polar and non-polar solvent. In an exemplary embodiment, the solvent includes but is not limited to ethyl acetate, methyl acetate, benzene, toluene, ethanol, acetic acid, acetonitrile, butanol, carbon tetrachloride, chlorobenzene, chloroform, cyclohexane, 1,2-dichloroethane, heptane, hexane, methanol, methylene chloride, nitromethane, pentane, propanol, and xylene. Alternatively, the process of preparing liquid salt of the present disclosure is carried out in absence of any solvent.

In an exemplary embodiment, the process of preparing adduct comprises act of contacting at least one electron-pair acceptor with at least one electron-pair donor in presence or absence of a solvent to obtain an adduct.

In an exemplary embodiment, the at least one electron-pair acceptor is contacted with the at least one electron-pair donor in presence or absence of a solvent under nitrogen atmosphere to obtain a slurry. The slurry is stirred and thereafter subjected to filtration, followed by washing with a solvent to obtain an adduct.

In an exemplary embodiment, the process of preparing adduct comprises acts of:
a) contacting at least one electron-pair acceptor with a first solvent under nitrogen atmosphere to obtain a mixture;
b) adding at least one electron-pair donor to the mixture of step (a) to obtain a slurry; and
c) stirring and filtering the slurry of step (b) and washing with a second solvent followed by drying to obtain an adduct.

In a non-limiting embodiment, the electron-pair acceptor or the Lewis acid employed in the process of preparing including but not limiting to adduct is present at an amount ranging from about 0.001 mol to about 0.9 mol, preferably about 0.3 mol to about 0.8 mol.

In a non-limiting embodiment, the electron-pair donor or the Lewis base employed in the process of preparing liquid salt including but not limiting to adduct is present at an amount ranging from about 0.001 mol to about 0.9 mol, preferably about 0.3 mol to about 0.8 mol.

In a non-limiting embodiment, the process of preparing the adduct comprises acts of:
a) contacting at least one electron-pair acceptor at an amount ranging from about 0.001 mol to about 0.9 mol with a first solvent under nitrogen atmosphere to obtain a mixture;
b) adding at least one electron-pair donor at an amount ranging from about 0.001 mol to about 0.9 mol to the mixture of step (a) under stirring to obtain a slurry; and
c) stirring and filtering the slurry of step (b) and washing with a second solvent followed by drying to obtain the adduct.

In an embodiment of the present disclosure, the first solvent is preferably ethyl acetate, methyl acetate, ethanol and methanol or any combination thereof. In an embodiment of the present disclosure, amount of the first solvent is ranging from about 1 w/w % to about 80 w/w %, preferably about 30 w/w % to about 50 w/w %.

In an embodiment of the present disclosure, the second solvent is preferably ethyl acetate, methyl acetate, ethanol, methanol and hexane or any combination thereof. In an embodiment of the present disclosure, amount of the second solvent is ranging from about 1 w/w % to about 80 w/w %, preferably about 5 w/w % to about 30 w/w %.

In an embodiment of the present disclosure, the third solvent is selected from group comprising benzene, toluene and xylene or any combination thereof. In an embodiment of the present disclosure, amount of the third solvent is ranging from about 1 w/w % to about 80 w/w %, preferably about 30 w/w % to about 70 w/w %.

In a non-limiting embodiment, the adduct of the method of the present disclosure is directly exposed to nitrogen atmosphere without the third solvent. Thus, in one embodiment, the electron-pair acceptor is added to the adduct in absence of any solvent.

In an embodiment of the present disclosure, liquid clathrate compounds are formed by interactions between aromatic solvent molecules and Ionic Liquid (ionic solid) ions, which separate cation-anion packing interactions to a sufficient degree such that localized cage-like structures are formed. If the interaction is very less, the ionic liquid is completely miscible/or immiscible with the aromatics compound. If the ion-ion interactions are very high, then crystallization of the salt/ionic liquid occurs. Thus, the liquid clathrate formation depends on the physical properties of the organic salts.

The present disclosure also relates to a salt prepared by the process of the present disclosure which involves reacting at least one electron-pair acceptor and at least one electron-pair donor to form an adduct, and further reacting the adduct with at least one electron-pair acceptor.

In a non-limiting embodiment, the present disclosure relates to a salt preferably liquid salt including but not limiting to ionic liquid prepared by the process of the present disclosure which involves reacting at least one electron-pair acceptor and at least one electron-pair donor to form an adduct, and further reacting the adduct with at least one electron-pair acceptor to prepare said liquid salt.

In an embodiment of the present disclosure, density of ionic liquid is measured by specific gravity method. In another embodiment of the present disclosure, viscosity of ionic liquid is measured by Oswald viscometer.

In an embodiment of the present disclosure, the ionic liquid is suitable for applications involving chemical reaction. In an embodiment of the present disclosure, the chemical reaction is an organic reaction.

In an exemplary embodiment, the liquid salt including but not limiting to ionic liquid is suitable for applications involving organic reactions including but not limiting to catalysis, alkylation, trans-alkylation, acylation, polymerization, dimerization, oligomerization, acetylation, metatheses, pericyclic and copolymerization reactions. In an exemplary embodiment, the liquid salt including but not limiting to ionic liquid is suitable for organic reaction including but not limiting to Diels-Alder reaction.

Additional embodiments and features of the present disclosure will be apparent to one of ordinary skill in art based upon description provided herein. The embodiments herein provide various features and advantageous details thereof in the description. Descriptions of well-known/conventional methods and techniques are omitted so as to not unnecessarily obscure the embodiments herein. The examples provided herein are intended merely to facilitate an understanding of ways in which the embodiments herein may be practiced and to further enable those of skill in the art to practice the embodiments herein. Accordingly, the following examples should not be construed as limiting the scope of the embodiments herein.

### Examples

### Example 1: Preparation of Ionic Liquid from DMSO-Aluminium Chloride Adduct

### (a) Preparation of DMSO-Aluminium Chloride Adduct

About 2.7 g (0.020 mol) of AlCl₃ and about 20 ml of ethyl acetate are charged into a 250 ml RB flask under N2 atmosphere. Slowly under stirring, about 5 g (0.064 mol) of DMSO is added for about 10 minutes at a temperature ranging from about 15° C. to about 20° C. to obtain a slurry. The whole mass is further stirred for about 4 hours. The resultant mixture is then separated by filtration. The solids obtained are washed with about 25 ml of fresh ethyl acetate followed by drying to get about 6.8 g of DMSO-Aluminium chloride adduct.

(b) Preparation of Ionic Liquid with Aluminium Chloride as the Electron Pair Acceptor (without Solvent)

About 15 g (0.040 mol) of DMSO-Aluminium chloride adduct obtained above is charged into a 100 ml single neck RB flask kept on a magnetic stirrer. N₂ flow is ensured inside the flask. The flask is kept in a water bath at a temperature ranging from about 30° C. to about 35° C. A magnetic needle is kept inside the flask for stirring. Slowly, about 32.7 g (0.245 mol) of AlCl₃ is added to the flask under stirring for about 30 minutes. The obtained mass is stirred for a time period ranging from about 3 hours to about 4 hours. The resultant ionic liquid is kept under nitrogen in closed conditions. Density of the IL is measured by specific gravity method and is found to be about 1.62 and its viscosity is measured by Oswald viscometer and is found to be about 220 cp.

(c) Preparation of Ionic Liquid with Aluminium Chloride as the Electron-Pair Acceptor in Presence of Solvent

About 15 g (0.040 mol) of DMSO-Aluminium chloride adduct obtained above and about 20 ml benzene are charged into a 100 ml single neck RB flask kept on a magnetic stirrer. N₂ flow is ensured inside the flask. The flask is kept in a water bath at a temperature ranging from about 30° C. to about 35° C. A magnetic needle is kept inside the flask for stirring. Slowly, about 32.7 g (0.245 mol) of AlCl₃ is added to the flask under stirring for about 30 minutes. The obtained mass is stirred for a time period ranging from about 3 hours to about 4 hours. The resultant ionic liquid is kept under nitrogen in closed conditions. Density of the IL obtained is about 1.40 and its viscosity is about 25 cp.

### Example 2: Preparation of Ionic Liquid from DMF-Aluminium Chloride Adduct

### (a) Preparation of DMF-Aluminium Chloride Adduct

About 2.7 g (0.020 mol) of AlCl₃ and about 20 ml of ethyl acetate are charged into a 250 ml RB flask under N₂ atmosphere. Slowly under stirring, about 4.5 g (0.063 mol) of DMF is added for about 10 minutes at a temperature ranging from about 15° C. to about 20° C. to obtain a mass. The whole mass is further stirred for about 4 hours. The resultant mixture is then separated by filtration. The solids obtained are washed with about 25 ml fresh ethyl acetate followed by drying to get about 6.5 g of DMF-Aluminium chloride adduct.

(b) Preparation of Ionic Liquid with Aluminium Chloride as the Electron Pair Acceptor (without Solvent)

About 14.1 g (0.040 mol) of DMF-Aluminium chloride adduct obtained above is charged into a 100 ml single neck RB flask kept on a magnetic stirrer. N₂ flow is ensured inside the flask. The flask is kept in a water bath at a temperature ranging from about 30° C. to about 35° C. A magnetic needle is kept inside the flask for stirring. Slowly, about 32.7 g (0.245 mol) of AlCl₃ is added to the flask under stirring for about 30 minutes. The obtained mass is stirred for a time period ranging from about 3 hours to about 4 hours. The resultant ionic liquid is kept under nitrogen in closed conditions.

(c) Preparation of Ionic Liquid with Aluminium Chloride as the Electron Pair Acceptor in Presence of Solvent

About 14.1 g (0.040 mol) of DMF-Aluminium chloride adduct obtained above and about 20 ml benzene are charged into a 100 ml single neck RB flask kept on a magnetic stirrer. N₂ flow is ensured inside the flask. The flask is kept in a water bath at a temperature ranging from about 30° C. to about 35° C. A magnetic needle is kept inside the flask for stirring. Slowly, about 32.7 g (0.245 mol) of AlCl₃ is added to the flask under stirring for about 30 minutes. The obtained mass is stirred for a time period ranging from about 3 hours to about 4 hours. The resultant ionic liquid is kept under nitrogen in closed conditions. Density of the IL obtained is about 1.45 and its viscosity is about 32 Cp.

### Example 3: Preparation of Ionic Liquid from IPA-Aluminium Chloride Adduct

### (a) Preparation of Isopropyl Alcohol-Aluminium Chloride Adduct

About 2.7 g (0.020 mol) of AlCl₃ and about 20 ml of ethyl acetate are charged into a 250 ml RB flask under N₂ atmosphere. Slowly under stirring, about 3.8 g (0.063 mol) of IPA is added for about 10 minutes at a temperature ranging from 15° C. to about 20° C. to obtain a mass. The whole mass is then stirred for about 4 hours. The resultant mixture is then separated by filtration. The solids obtained are washed with about 25 ml of fresh ethyl acetate followed by drying to get about 5.04 g of IPA-Aluminium chloride adduct.

(b) Preparation of Ionic Liquid with Aluminium Chloride as the Electron Pair Acceptor

About 12.53 g (0.040 mol) of IPA-Aluminium chloride adduct obtained above and about 20 ml benzene are charged into a 100 ml single neck RB flask kept on a magnetic stirrer. N₂ flow is ensured inside the flask. The flask is kept in a water bath at a temperature ranging from about 30° C. to about 35° C. A magnetic needle is kept inside the flask for stirring. Slowly, about 32.7 g (0.245 mol) of AlCl₃ is added to the flask under stirring for about 30 minutes. The obtained mass is stirred for a time period ranging from about 3 hours to about 4 hours. The resultant ionic liquid is kept under nitrogen in closed conditions.

### Example 4: Preparation of Ionic Liquid from DMSO-Aluminium Chloride Adduct and Zinc Chloride as the Electron-Pair Acceptor

### (a) Preparation of DMSO-Aluminium Chloride Adduct

The DMSO-Aluminium chloride adduct is prepared on the basis of the process described in the present disclosure and protocols of examples including but not limiting to Example 1 and Example 3 above.

(b) Preparation of Ionic Liquid with Zinc Chloride as the Electron-Pair Acceptor

About 14.7 g (0.04 mol) of DMSO-Aluminium chloride adduct is charged into a 100 ml glass reactor kept under an overhead stirrer, placed in a water bath at 30-35° C. Then, about 32.7 g (0.24 mol) of Zinc Chloride (ZnCl₂) is slowly added in to it with constant stirring. N₂ flow is ensured inside the reactor. The mixture is stirred for about 3 hours to get ionic liquid.

### Example 5: Preparation of Ionic Liquid from DMSO-Aluminium Chloride Adduct and Ferric Chloride as the Electron-Pair Acceptor

### (a) Preparation of DMSO-Aluminium Chloride Adduct

About 22.8 g (0.17 mol) of AlCl₃ and about 100 ml of ethanol are charged into a 250 ml glass reactor kept under an overhead stirrer, placed in a water bath. Then, about 40.9 g (0.17 mol) of DMSO is slowly added in to it with constant stirring. N₂ flow is ensured inside the reactor. The mixture is stirred for about 4 h to get white coloured solid. The reaction mass is allowed to settle for about 10 minutes. The solid is then separated and dried at about 100° C. to obtain DMSO-Aluminium chloride adduct.

(b) Preparation of Ionic Liquid with Ferric Chloride as the Electron-Pair Acceptor

About 14.7 g (0.04 mol) of DMSO-Aluminium chloride adduct is charged into a 100 ml glass reactor kept under an overhead stirrer, placed in a water bath at 30-35° C. Then, about 39 g (0.24 mol) of Ferric Chloride (FeCl₃) is slowly added in to it with constant stirring. N₂ flow is ensured inside the reactor. The mixture is stirred for about 3 hours to get ionic liquid.

### Example 6: Preparation of Ionic Liquid from Urea-Aluminium Chloride Adduct and Aluminium Chloride as the Electron-Pair Acceptor

### (a) Preparation of Urea-Aluminum Chloride Adduct

13.84 g (0.22 mol) of urea and about 60 ml of ethanol are charged into a 250 ml RB flask under N₂ atmosphere for 1-2 hrs. Slowly under stirring, about 9.62 g (0.072 mol) of AlCl₃ is added for about 30 minutes at about 15-20° C. to obtain a mass. The whole mass is then stirred for about 4 hours. The resultant mixture is then separated by filtration. The solids are washed with about 50 ml fresh ethanol followed by drying to get about 22.3 g of urea-Aluminum chloride salt.

AlCl₃+3Urea→[Al Urea₃Cl₃]

(b) Preparation of Ionic Liquid with Aluminium Chloride as the Electron-Pair Acceptor (with Solvent)

About 10 g (0.030 mol) of total solid powder obtained in the Example 6-a and about 20 ml benzene are charged into a 100 ml single neck RB flask kept on a magnetic stirrer. N₂ flow is ensured inside the flask. The flask is kept in a water bath at about 30-35° C. A magnetic needle is kept inside the flask for stirring. Slowly, about 24.4 g (0.18 mol) of AlCl₃ is added to the flask under stirring for about 30 minutes. The obtained mass is stirred for about 3-4 hours at room temperature. Ionic Liquid formed is stored in inert atmosphere. The density of the IL is 1.25 and the viscosity is 9 Cp.

(c) Preparation of Ionic Liquid with Aluminium Chloride as the Electron Pair Acceptor (without Solvent)

About 10 g (0.030 mol) of total solid powder obtained in the Example 6-a is charged into a 100 ml single neck RB flask kept on a magnetic stirrer. N₂ flow is ensured inside the flask. The flask is kept in a water bath at about 30-35° C. A magnetic needle is kept inside the flask for stirring. Slowly, about 24.4 g (0.18 mol) of AlCl₃ is added to the flask under stirring for about 30 minutes. The obtained mass is stirred for about 3-4 h. The Ionic Liquid formed is stored in inert atmosphere. The density of the IL is 1.61.

### Example 7: Preparation of Ionic Liquid from DMF-Aluminium Chloride Adduct and Ferric Chloride as the Electron-Pair Acceptor

### (a) Preparation of DMF-Aluminium Chloride Adduct

About 2.7 g (0.020 mol) of AlCl₃ and about 20 ml of ethyl acetate are charged into a 250 ml RB flask under N₂ atmosphere. Slowly under stirring, about 4.65 g (0.063 mol) of DMF is added for about 10 minutes at a temperature ranging from about 15-20° C. to obtain a mass. The whole mass is then stirred for about 4 hrs. The resultant mixture is separated by filtration and the solid obtained is washed with about 25 ml fresh ethyl acetate followed by drying at about 100° C. to get about 6.5 g of DMF-Aluminium chloride adduct.

(b) Preparation of Ionic Liquid with Ferric Chloride as the Electron-Pair Acceptor

About 14.2 g (0.04 mol) of DMF-Aluminium chloride adduct is charged into a 100 ml glass reactor kept under an overhead stirrer, placed in a water bath kept at 30-35° C. Then, about 38.9 g (0.24 mol) of Ferric Chloride (FeCl₃) is slowly added in to it with constant stirring. N₂ flow is ensured inside the reactor. The mixture is stirred for about 3 hours to get ionic liquid.

### Example 8: Preparation of Ionic Liquid from N-Methylpyrolidone-Aluminium Chloride Adduct and Aluminium Chloride as the Electron-Pair Acceptor

### (a) Preparation of N-Methylpyrolidone-Aluminium Chloride Adduct

About 13.36 g (0.1 mol) of anhydrous AlCl3 is added under slow stirring to about 10 g (0.1 mol) of N-methylpyrolidone in a round bottom flask and the mixture is kept in water bath at room temperature for about 240 minutes. The two reactants are mixed, and an exothermic reaction is observed wherein they fuse to form a white solid adduct of N-methylpyrolidone-Aluminium chloride (1:1 adduct).

(b) Preparation of Ionic Liquid with Aluminium Chloride as the Electron Pair Acceptor

About 5 (0.021 mol) g of solid adduct of N-methylpyrolidone-Aluminium chloride is mixed with about 2.86 (0.021 mol) g of Aluminium chloride in RB Flask with nitrogen purge at room temperature. The above mixture is stirred for about 3.5 h and the resulting homogenous eutectic liquid formed is an ionic liquid compound.

### Example 9: Preparation of Ionic Liquid from NMP-Aluminium Chloride Adduct

### (a) Preparation of NMP-Aluminium Chloride Adduct

About 13.36 g (0.1 mol) of AlCl3 and about 20 ml of ethyl acetate are charged into a 250 ml RB flask under N₂ atmosphere. Slowly under stirring, about 10 g (0.1 mol) of NMP is added for about 10 minutes at a temperature ranging from about 15° C. to about 20° C. to obtain a slurry. The whole mass is further stirred for about 4 hours. The resultant mixture is then separated by filtration. The solids obtained are washed with about 25 ml of fresh ethyl acetate followed by drying to get about 20 g of NMP-Aluminium chloride adduct (1:1 adduct).

(b) Preparation of Ionic Liquid with Aluminium Chloride as the Electron Pair Acceptor (without Solvent)

About 5 g (0.021 mol) of NMP-Aluminium chloride adduct obtained above is charged into a 100 ml single neck RB flask kept on a magnetic stirrer. N₂ flow is ensured inside the flask. The flask is kept in a water bath at a temperature ranging from about 30° C. to about 35° C. A magnetic needle is kept inside the flask for stirring. Slowly, about 2.86 g (0.021 mol) of AlCl₃ is added to the flask under stirring for about 30 minutes. The obtained mass is stirred for a time period ranging from about 3 hour to about 4 hours. The resultant ionic liquid is kept under nitrogen in closed conditions.

(c) Preparation of Ionic Liquid with Aluminium Chloride as the Electron-Pair Acceptor in Presence of Solvent

About 5 g (0.021 mol) of NMP-Aluminium chloride adduct obtained above and about 5 ml benzene are charged into a 100 ml single neck RB flask kept on a magnetic stirrer. N₂ flow is ensured inside the flask. The flask is kept in a water bath at a temperature ranging from about 30° C. to about 35° C. A magnetic needle is kept inside the flask for stirring. Slowly, about 2.86 g (0.021 mol) of AlCl₃ is added to the flask under stirring for about 30 minutes. The obtained mass is stirred for a time period ranging from about 3 hour to about 4 hours. The resultant ionic liquid is kept under nitrogen in closed conditions.

### Example 10

### (a) Preparation of Electron-Pair Acceptor-Electron-Pair Donor Adduct

About 13.35 g (0.1 mol) of AlCl3 and about 20 ml of ethyl acetate are charged into a 250 ml RB flask under N2 atmosphere. Slowly under stirring, about 22.2 g (0.3 mol) of diethyl ether is added for about 10 minutes at a temperature ranging from about 15° C. to about 20° C. to obtain a slurry. The whole mass is further stirred for about 4 hours. The resultant mixture is then separated by filtration. The solids obtained are washed with about 25 ml of fresh ethyl acetate followed by drying to get about 20 g of diethyl ether-aluminium chloride adduct the adduct formed here is (1:1 adduct).

Similarly, about 19 g of tetrahydrofuran-aluminium chloride adduct is formed by using about 13.35 g (0.1 mol) of AlCl3 and 21.6 g (0.3 mol) of tetrahydrofuran as the electron-pair donor instead of diethyl ether. The adduct formed in this case is also 1:1 adduct.

Similarly, about 18 g of ethylene glycol-aluminium chloride adduct is formed by using about 13.35 g (0.1 mol) of AlCl3 and 6.2 g (0.1 mol) of ethylene glycol as the electron-pair donor instead of diethyl ether. The adduct formed in this case is also 1:1 adduct.

Similarly, about 22 g of glycerol-aluminium chloride adduct is formed by using about 13.35 g (0.1 mol) of AlCl3 and 9.2 g (0.1 mol) of glycerol as the electron-pair donor instead of diethyl ether. The adduct formed in this case is also 1:1 adduct.

Similarly, about 20 g of propylene glycol-aluminium chloride adduct is formed by using about 13.35 g (0.1 mol) of AlCl3 and 7.6 g (0.1 mol) of propylene glycol as the electron-pair donor instead of diethyl ether. The adduct formed in this case is also 1:1 adduct.

Similarly, about 21 g of cyclopentanone-aluminium chloride adduct is formed by using about 13.35 g (0.1 mol) of AlCl3 and 8.4 g (0.1 mol) of cyclopentanone as the electron-pair donor instead of diethyl ether. The adduct formed in this case is also 1:1 adduct.

Similarly, about 22.5 g of cyclohexanone-aluminium chloride adduct is formed by using about 13.35 g (0.1 mol) of AlCl3 and 9.8 g (0.1 mol) of cyclohexanone as the electron-pair donor instead of diethyl ether. The adduct formed in this case is also 1:1 adduct.

(b) Preparation of Ionic Liquid with Addition of Electron Pair Acceptor

About 10 g (0.048 mol) of the adducts obtained above are charged individually into 100 ml single neck RB flask kept on a magnetic stirrer. N₂ flow is ensured inside the flask. The flask is kept in a water bath at a temperature ranging from about 30° C. to about 35° C. A magnetic needle is kept inside the flask for stirring. Slowly, about 33.7 g (0.29 mol) of AlCl₃ is added to the flask under stirring for about 30 minutes. The obtained mass is stirred for a time period ranging from about 3 hour to about 4 hours. However, no ionic liquid is formed and the mixture remained as a solid. All the above compounds are found to form a stable adduct with AlCl₃ in a 1:1 ratio in first step (step a) and do not form ionic liquid in second step (step b). Addition of extra reagent in step a) or step b) does not make any difference.

### Example 11: Alkylation Reaction by Ionic Liquid from DMSO-Aluminium Chloride Adduct (Prepared in Example 1)

About 52.02 litres of hydrocarbon stream containing about 10% to about 13% of C10-C14 olefins and about 87% to about 90% paraffins and about 20.02 litres of benzene are charged into a 250 L glass reactor kept under an overhead stirrer, placed in a heating mantle. N2 flow is ensured inside the reactor. The reactor is then heated to a temperature ranging from about 38° C. to about 39° C. Once the temperature is achieved, about 0.7 kg of the ionic liquid catalyst prepared as per Example 1 is added to the reactor and stirred for about 5 minutes. After about 5 minutes, the reaction mass is allowed to settle for about 10 minutes. The layers are then separated.

The upper hydrocarbon layer is then analysed using titration. The conversion of olefins is found to be 98% to form linear alkyl benzene.

The lower layer is recycled with fresh hydrocarbon stream and benzene as per the procedure above. The conversion of olefins present in the paraffin stream to linear alkyl benzene is analysed and is found to be about 98%.

### Example 12: Alkylation Reaction by Ionic Liquid from DMSO-Aluminium Chloride Adduct (Prepared in Example 1)

About 141.5 ml (124.3 gm) of benzene is added to a 250 ml RB flask kept under an overhead stirrer under N2 atm. About 7.5 g of ionic liquid catalyst prepared as per Example 1 is added to the flask. About 23.4 ml benzyl chloride is added to the flask at a temperature ranging from about 45° C. to about 46° C. and stirred for about 15 minutes. After completion of reaction, catalyst and hydrocarbon layers are separated. The upper hydrocarbon layer is then analysed by gas chromatography for benzyl chloride conversion. The conversion of benzyl chloride to biphenyl methane is analysed by gas chromatography and is found to be about 92%.

### Example 13: Oligomerization Reaction by Ionic Liquid from DMSO-Aluminium Chloride Adduct (Prepared in Example 1b)

About 100 ml of hydrocarbon stream containing about 10% to about 13% of C₁₀-C₁₄ olefins and about 87% to about 90% of paraffins are charged into a 250 ml glass reactor kept under an overhead stirrer, placed in a heating mantle. N₂ flow is ensured inside the reactor. The reactor is then heated to about 45° C. Once the temperature is achieved, about 0.1 g of the ionic liquid catalyst prepared as per Example 1 is added to the reactor and stirred for about 10 minutes. After about 10 minutes the reaction mass is allowed to settle for about 10 minutes. The layers are then separated. The upper hydrocarbon layer is then analysed. The conversion of olefins is analysed using titration and is found to be about 97%.

### Example 14: Alkylation Reaction by Ionic Liquid from DMSO-Aluminium Chloride Adduct (Prepared in Example 1b)

About 23.5 g of Phenol and about 2.2 g of Methyl tert-butyl ether (MTBE) are charged into a 100 ml glass reactor kept under an overhead stirrer, placed in a heating mantle. N₂ flow is ensured inside the reactor. The reactor is then heated to a temperature of about 60° C. Once the temperature is achieved, about 0.25 g of the ionic liquid catalyst prepared as per Example 1 is added to the reactor and stirred for about 3 hours. After about 3 hrs the reaction is worked-up with 25 ml distilled water. The conversion of MTBE is analysed and is found to be about 95%.

### Example 15: Diels-Alder Reaction by Ionic Liquid from DMSO-Aluminium Chloride Adduct (Prepared in Example 1b)

About 2.76 g of Isoprene and about 1.02 g Vinyl Acetate are charged into a 100 ml glass reactor kept under an overhead stirrer, placed in a heating mantle. N₂ flow is ensured inside the reactor. The reactor is then heated to a temperature of about 60° C. Once the temperature is achieved, about 0.03 g of the ionic liquid catalyst prepared as per Example 1 is added to the reactor and stirred for about 4 hours. After about 4 hours the reaction is worked-up with 10 ml ethyl acetate. The conversion of reactants is analysed and is found to be about 96%.

### Example 16: Acylation Reaction by Ionic Liquid from DMSO-Aluminium Chloride Adduct (Prepared in Example 1)

About 19.5 g of Benzene and about 3.5 g Acetyl Chloride are charged into a 100 ml glass reactor kept under an overhead stirrer, placed in a heating mantle. N₂ flow is ensured inside the reactor. The reactor is then heated to a temperature of about 60° C. Once the temperature is achieved, about 0.21 g of the ionic liquid catalyst prepared as per Example 1 is added to the reactor and stirred for about 2 hrs. After about 2 hours, the reaction is worked-up with about 25 ml distilled water. The conversion of Acetyl Chloride is analysed and is found to be about 96%.

### Example 17: Acylation Reaction by Ionic Liquid from DMSO-Aluminium Chloride Adduct (Prepared in Example 1)

About 19.5 g of Benzene and about 1.95 g Benzoyl Chloride are charged into a 100 ml glass reactor kept under an overhead stirrer, placed in a heating mantle. N₂ flow is ensured inside the reactor. The reactor is then heated to a temperature of about 60° C. Once the temperature is achieved, about 0.21 g of the ionic liquid catalyst prepared as per Example 1 is added to the reactor and stirred for about 3 hours. The reaction is worked-up with about 15 ml distilled water and 15 ml of ethyl acetate. The conversion of Benzoyl Chloride is analysed and is found to be 91%.

The present disclosure in view of the above described illustrations and various embodiments, is thus able to successfully overcome the various deficiencies of prior art and provide for an improved process for preparing liquid salt including but not limiting to ionic liquid.

Additional embodiments and features of the present disclosure will be apparent to one of ordinary skill in art based on the description provided herein. The embodiments herein provide various features and advantageous details thereof in the description. Descriptions of well-known/conventional methods and techniques are omitted so as to not unnecessarily obscure the embodiments herein.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments in this disclosure have been described in terms of preferred embodiments, those skilled in the art will recognize that the embodiments herein can be practiced with modification within the spirit and scope of the embodiments as described herein.

Throughout this specification, the word “comprise”, or variations such as “comprises” or “comprising” wherever used, will be understood to imply the inclusion of a stated element, integer or step, or group of elements, integers or steps, but not the exclusion of any other element, integer or step, or group of elements, integers or steps.

The use of the expression “at least” or “at least one” suggests the use of one or more elements or ingredients or quantities, as the use may be in the embodiment of the disclosure to achieve one or more of the desired objects or results.

Any discussion of documents, acts, materials, devices, articles and the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

While considerable emphasis has been placed herein on the particular features of this disclosure, it will be appreciated that various modifications can be made, and that many changes can be made in the preferred embodiments without departing from the principles of the disclosure. These and other modifications in the nature of the disclosure or the preferred embodiments will be apparent to those skilled in the art from the disclosure herein, whereby it is to be distinctly understood that the foregoing descriptive matter is to be interpreted merely as illustrative of the disclosure and not as a limitation.

1) A method of preparing ionic liquid, said method comprising acts of:
a) contacting at least one electron-pair acceptor with at least one electron-pair donor to obtain an adduct; and
b) contacting the adduct with at least one electron-pair acceptor to obtain the ionic liquid.

2) An ionic liquid prepared according to the method of claim 1.

3) A method of using the ionic liquid as claimed in claim 2 for application in chemical reaction.

4) A method of preparing an adduct of electron-pair acceptor and electron-pair donor, said method comprising act of contacting at least one electron-pair acceptor with at least one electron-pair donor to obtain the adduct.

5) An adduct prepared accordingly to the method of claim 4.

6) The method as claimed in claim 1, wherein the method of preparing the ionic liquid comprises acts of:
a) contacting at least one electron-pair acceptor with at least one electron-pair donor, in presence or absence of first solvent, to obtain a mixture;
b) optionally mixing and filtering the mixture of step (a) to obtain a filtrate and optionally washing the filtrate or the mixture of step (a) with a second solvent, followed by drying to obtain the adduct; and
c) contacting the adduct of step (b) with at least one electron-pair acceptor in presence or absence of third solvent, followed by mixing to obtain the ionic liquid.

7) The method as claimed in claim 4, wherein the method of preparing the adduct comprises acts of:
a) contacting at least one electron-pair acceptor with at least one electron-pair donor, in presence or absence of first solvent, to obtain a mixture; and
b) optionally mixing and filtering the mixture of step (a) to obtain a filtrate and optionally washing the filtrate or the mixture of step (a) with a second solvent, followed by drying to obtain the adduct.

8) The method as claimed in claim 1, wherein the step a) or step b) or a combination thereof is carried out in presence of solvent; the electron acceptor used in step b) is same as or different than that used in step a); addition of the solvent is carried out along with mixing; ratio of the electron-pair acceptor to the electron-pair donor in step a) is ranging from about 1:1 to about 1:5; concentration of the adduct in step b) is ranging from about 0.001 mol to about 0.9 mol; and ratio of the adduct to the electron-pair acceptor in step b) is ranging from about 1:1 to about 1:6.

9) The method as claimed in claim 4, wherein the method is carried out in presence of solvent; addition of the solvent is carried out along with mixing; and ratio of the electron-pair acceptor to the electron-pair donor is ranging from about 1:1 to about 1:5.

10) The method as claimed in claim 1, wherein the method of preparing the ionic liquid is carried out in absence of heating; the method is carried out under inert atmosphere; and wherein the inert atmosphere is Nitrogen atmosphere.

11) The method as claimed in claim 1, wherein the electron acceptor is a salt of cation selected from group comprising aluminium, magnesium, calcium, chromium, manganese, iron, cobalt, nickel, copper, zinc, gallium, germanium, zirconium, scandium, vanadium, molybdenum, ruthenium, rhodium, indium, tin, titanium, lead, cadmium and mercury or any combination thereof; the electron acceptor is a salt of cation selected from group comprising acetate, carbonate, chloride, citrate, cyanide, fluoride, nitrate, nitrite, phosphate and sulfate or any combination thereof; and the concentration of the electron acceptor is ranging from about 0.001 mol to about 0.9 mol.

12) The method as claimed in claim 1, wherein the electron donor is not an amine; the electron donor is selected from group comprising phosphine, amide, alkyl sulfoxide, ester and alcohol or any combination thereof; the phosphine is selected from group comprising triphenylphosphine, triphenylphosphine oxide, trimethylphosphine and tributylphosphine or any combination thereof; the amide is selected from group comprising urea, dimethyl formamide, acetamide, N-methyl pyrrolidine, thiourea, phenylthiourea, acetanilide, propanamide, 3-methylbutanamide, dimethylacetamide and butanamide or any combination thereof; the alkyl sulfoxide is dimethyl sulfoxide; the ester is selected from group comprising amyl acetate, ethyl acetate and propyl acetate or any combination thereof; the alcohol is cyclohexanol and isopropyl alcohol or any combination thereof; and the concentration of the electron donor is ranging from about 0.001 mol to about 0.9 mol.

13) The method as claimed in claim 1, wherein the first solvent, the second solvent or the third solvent are same or different; the solvent is selected from group comprising Ethyl Acetate, Methyl Acetate, Benzene, Toluene, Ethanol, Acetic Acid, Acetonitrile, Butanol, Carbon Tetrachloride, Chlorobenzene, Chloroform, Cyclohexane, 1,2-Dichloroethane, Heptane, Hexane, Methanol, Methylene Chloride, Nitromethane, Pentane, Propanol and Xylene or any combination thereof; and the amount of the solvent is ranging from about 1% to about 80%.

14) The method as claimed in claim 6, wherein the first solvent, the second solvent or the third solvent are same or different; the solvent is selected from group comprising Ethyl Acetate, Methyl Acetate, Benzene, Toluene, Ethanol, Acetic Acid, Acetonitrile, Butanol, Carbon Tetrachloride, Chlorobenzene, Chloroform, Cyclohexane, 1,2-Dichloroethane, Heptane, Hexane, Methanol, Methylene Chloride, Nitromethane, Pentane, Propanol and Xylene or any combination thereof; the amount of the solvent is ranging from about 1% to about 80%; the solvent in step a) is added to either the electron-pair acceptor or the electron-pair donor, prior to the said contacting; the contacting is carried out along with mixing; the mixing is carried out for a time duration ranging from about 1 minute to about 12 hours, at a temperature ranging from about 5° C. to about 50° C.; and the mixing is carried out by technique selected from group comprising stirring, milling, blending, static mixing, and grinding, or any combination thereof.

15) The method as claimed in claim 3, wherein the chemical reaction is selected from group comprising catalysis, alkylation reaction, trans-alkylation reaction, acylation reaction, polymerization reaction, dimerization reaction, oligomerization reaction, acetylation reaction, metatheses reaction, pericyclic reaction and copolymerization reaction or any combination thereof.

16) The method as claimed in claim 4, wherein the method of preparing the ionic liquid is carried out in absence of heating; the method is carried out under inert atmosphere; and wherein the inert atmosphere is Nitrogen atmosphere.

17) The method as claimed in claim 4, wherein the electron acceptor is a salt of cation selected from group comprising aluminium, magnesium, calcium, chromium, manganese, iron, cobalt, nickel, copper, zinc, gallium, germanium, zirconium, scandium, vanadium, molybdenum, ruthenium, rhodium, indium, tin, titanium, lead, cadmium and mercury or any combination thereof; the electron acceptor is a salt of cation selected from group comprising acetate, carbonate, chloride, citrate, cyanide, fluoride, nitrate, nitrite, phosphate and sulfate or any combination thereof; and the concentration of the electron acceptor is ranging from about 0.001 mol to about 0.9 mol.

18) The method as claimed in claim 4, wherein the electron donor is not an amine; the electron donor is selected from group comprising phosphine, amide, alkyl sulfoxide, ester and alcohol or any combination thereof; the phosphine is selected from group comprising triphenylphosphine, triphenylphosphine oxide, trimethylphosphine and tributylphosphine or any combination thereof; the amide is selected from group comprising urea, dimethyl formamide, acetamide, N-methyl pyrrolidine, thiourea, phenylthiourea, acetanilide, propanamide, 3-methylbutanamide, dimethylacetamide and butanamide or any combination thereof; the alkyl sulfoxide is dimethyl sulfoxide; the ester is selected from group comprising amyl acetate, ethyl acetate and propyl acetate or any combination thereof; the alcohol is cyclohexanol and isopropyl alcohol or any combination thereof; and the concentration of the electron donor is ranging from about 0.001 mol to about 0.9 mol.

19) The method as claimed in claim 4, wherein the first solvent, the second solvent or the third solvent are same or different; the solvent is selected from group comprising Ethyl Acetate, Methyl Acetate, Benzene, Toluene, Ethanol, Acetic Acid, Acetonitrile, Butanol, Carbon Tetrachloride, Chlorobenzene, Chloroform, Cyclohexane, 1,2-Dichloroethane, Heptane, Hexane, Methanol, Methylene Chloride, Nitromethane, Pentane, Propanol and Xylene or any combination thereof; and the amount of the solvent is ranging from about 1% to about 80%.

20) The method as claimed in claim 7, wherein the solvent in step a) is added to either the electron-pair acceptor or the electron-pair donor, prior to the said contacting; the contacting is carried out along with mixing; the mixing is carried out for a time duration ranging from about 1 minute to about 12 hours, at a temperature ranging from about 5° C. to about 50° C.; and the mixing is carried out by technique selected from group comprising stirring, milling, blending, static mixing, and grinding, or any combination thereof.
